SEQUENCE LISTING (12 sequences)

SEQ ID NO: 1 - DNA, 25 bp, Artificial Sequence
  KRAS codon 12-13 forward primer
ctgaatataaacttgtggtagttgg

SEQ ID NO: 2 - DNA, 20 bp, Artificial Sequence
  KRAS codon 12-13 reverse primer
gtcctgcaccagtaatatgc

SEQ ID NO: 3 - DNA, 11 bp, Artificial Sequence
  KRAS codon 12-13 blocker nucleic acid fragment
  misc_feature (1)..(3): BNA
  misc_feature (5)..(5): BNA
  misc_feature (7)..(8): BNA
  misc_feature (10)..(11): BNA
cctacgccacc

SEQ ID NO: 4 - DNA, 27 bp, Artificial Sequence
  KRAS codon 12-13 probe
  misc_feature (1)..(1): FAM
  misc_feature (27)..(27): TAMRA
tgccttgacgatacagctaattcagaa

SEQ ID NO: 5 - DNA, 20 bp, Artificial Sequence
  KRAS codon 61 forward primer
actgtgtttctcccttctca

SEQ ID NO: 6 - DNA, 20 bp, Artificial Sequence
  KRAS codon 61 reverse primer
cagtcctcatgtactggtcc

SEQ ID NO: 7 - DNA, 14 bp, Artificial Sequence
  KRAS codon 61 blocker nucleic acid fragment
  misc_feature (1)..(7): BNA
  misc_feature (10)..(11): BNA
  misc_feature (14)..(14): BNA
tcctcttgacctgc

SEQ ID NO: 8 - DNA, 25 bp, Artificial Sequence
  KRAS codon 61 probe
  misc_feature (1)..(1): FAM
  misc_feature (25)..(25): TAMRA
tccaagagacaggtttctccatcaa

SEQ ID NO: 9 - DNA, 22 bp, Artificial Sequence
  KRAS codon 146 forward primer
gtgatttgccttctagaacagt

SEQ ID NO: 10 - DNA, 27 bp, Artificial Sequence
  KRAS codon 146 reverse primer
cagaaaacagatctgtatttatttcag

SEQ ID NO: 11 - DNA, 12 bp, Artificial Sequence
  KRAS codon 146 blocker nucleic acid fragment
  misc_feature (1)..(5): BNA
  misc_feature (7)..(8): BNA
  misc_feature (11)..(11): BNA
tctttgctgatg

SEQ ID NO: 12 - DNA, 28 bp, Artificial Sequence
  KRAS codon 146 probe
  misc_feature (1)..(1): FAM
  misc_feature (28)..(28): TAMRA
aggctcaggacttagcaagaagttatgg

(19) United States
(12) Patent Application Publication
(10) Pub. No.: US 2017/0275675 A1
(43) Pub. Date: Sep. 28, 2017

(54) DETECTION METHOD AND KIT OF BASE MUTATION, AND METHOD FOR LIMITING PCR AMPLIFICATION OF NUCLEIC ACID SAMPLE

(71) Applicants: TOPPAN PRINTING CO., LTD., Tokyo (JP); RIKEN GENESIS CO., LTD., Tokyo (JP)

(72) Inventors: Shun SAKAMOTO, Tokyo (JP); Junko NAKAMURA, Tokyo (JP)

(73) Assignees: TOPPAN PRINTING CO., LTD., Tokyo (JP); RIKEN GENESIS CO., LTD., Tokyo (JP)

(21) Appl. No.: 15/617,680

(22) Filed: Jun. 8, 2017

Related U.S. Application Data
(63) Continuation of application No. PCT/JP2015/084743, filed on Dec. 11, 2015.

(30) Foreign Application Priority Data
Dec. 11, 2014 (JP) 2014-250901

(51) Int. Cl. C12Q 1/68

(52) U.S. Cl. CPC C12Q 1/686; C12Q 1/6806; C12Q 1/6853; C12Q 1/6837; C12Q 1/6869; C12Q 1/68

(57) ABSTRACT

Detection method of a base mutation in a target base sequence of a nucleic acid sample, includes: performing a PCR reaction with the nucleic acid sample as a template, using a primer set capable of amplifying, by PCR, an amplification target region including the target base sequence; a blocker nucleic acid fragment having a base sequence complementary to the target base sequence and including a residue which is synthetic nucleic acid; and a probe that hybridizes to a region, in the target region, closer to a 5′ end of the target region than the target base sequence in the same chain as the target base sequence, and that has a fluorescent substance on one of a 5′ end and a 3′ end of the probe and a quenching substance on the other; and measuring an amplification amount of the template in the PCR reaction by detecting fluorescence from the probe.

### CROSS REFERENCE TO RELATED APPLICATIONS

This application is a continuation application based on a PCT Patent Application No. PCT/JP2015/084743, filed Dec. 11, 2015, whose priority is claimed on Japanese Patent Application No. 2014-250901, filed Dec. 11, 2014, the entire contents of which are hereby incorporated by reference.

### BACKGROUND OF THE INVENTION

Field of the Invention

The present invention relates to detection method and kit of a base mutation, and a method for sequence-specifically limiting PCR amplification of a nucleic acid sample.

Description of the Related Art

The base sequences of genes vary between individuals. Of these differences in base sequences, those having a frequency of 1% or more are known as “gene polymorphisms”. There are various types of polymorphisms, including base substitutions, base insertions or deletions, differences in the number of times a specific base sequence is repeated, and the like. These polymorphisms are known as base mutations.

Base mutations can be detected by a method that involves analyzing the melting curves of polymerase chain reaction (PCR) products, a TaqMan (registered trademark) method, a method that involves sequencing the base sequences of PCR products, an Invader (registered trademark) method, or the like.

In the method that involves analyzing melting curves, the vicinity of the target base sequence that is to be detected is amplified by PCR. PCR is a method that involves using a heat-resistant polymerase and two primers that are complementary to the target nucleic acid, and controlling the temperature so as to repeat the three steps of (1) denaturation of a double-stranded target nucleic acid to be used as a template, (2) annealing of the primers to the denatured target nucleic acid, and (3) elongation from the primers, thereby exponentially amplifying the target nucleic acid.

Next, an intercalator reagent is used to prepare the melting curve of the PCR product. The intercalator reagent, when present in a solution, is present as an aggregate of two or more molecules, and therefore will not exhibit fluorescence due to excitonic effects. However, when intercalated into a target nucleic acid, the reagent is isolated from the aggregate state, and will then fluoresce. Examples of intercalator reagents include ethidium bromide and SYBR green.

More specifically, as the temperature of the PCR product is raised and the double-stranded DNA is separated (melted) into single strands of DNA, the fluorescence from the intercalator reagent will be quenched. A melting curve is prepared by making use of this effect. Subsequently, the melting curve is analyzed, and base mutations are detected on the basis of slight differences in the melting curves due to differences in the base sequences of the PCR products.

Additionally, the TaqMan (registered trademark) method involves using a TaqMan (registered trademark) probe, to which a fluorescent substance and a quenching substance are attached. When a PCR reaction is performed in the presence of a TaqMan (registered trademark) probe, the TaqMan (registered trademark) probe is annealed to the target nucleic acid during step (2) in the PCR method explained above. Furthermore, during the elongation reaction in step (3), the TaqMan (registered trademark) probe is degraded by the 5′-to-3′ exonuclease activity of the polymerase. As a result thereof, the fluorescent substance that has been freed from the quenching substance begins to fluoresce.

When detecting base mutations by means of the TaqMan (registered trademark) method, a PCR reaction is performed in the presence of two types of TaqMan (registered trademark) probes, i.e. a TaqMan (registered trademark) probe that is complementary to a sequence having a base mutation (mutant sequence) and a TaqMan (registered trademark) probe that has a different fluorescent substance from the aforementioned TaqMan (registered trademark) probe and that is complementary to the wild-type sequence. Thereafter, base mutations are detected by comparing the fluorescence intensities of the two colors of fluorescence from the respective TaqMan (registered trademark) probes that have degraded.

Additionally, in the sequencing method, base mutations are detected by directly decoding the base sequence of the PCR product.

Additionally, in the Invader (registered trademark) method, base mutations are detected by using a cleavase that specifically recognizes and cleaves a triple-stranded structure in the DNA.

Of these base sequence detection methods, the sequencing method and the Invader (registered trademark) method are better capable of detecting single-base mutations than the detection method using melting curves and the TaqMan (registered trademark) method.

Meanwhile, among base mutations, there are somatic cell mutations in which a mutation in the base sequence is acquired by a disease such as cancer. The detection of the presence or absence of somatic cell mutations is very useful when choosing treatment methods for diseases.

Somatic cell mutations include many unknown mutant sequences. Therefore, there is a problem in that, in order to detect these base mutations by a sequencing method or the Invader (registered trademark) method, the primer or probe sequence that is used must be reconsidered each time a new base mutation is discovered.

Additionally, in the case of somatic cell mutations, a nucleic acid sample will mostly contain the wild-type sequence, and there may be just a few copies of nucleic acids having the mutant sequence. In this case, it may be difficult to detect somatic cell mutations by using the detection method using melting curves or the TaqMan (registered trademark) method, which have low detection sensitivity.

Therefore, a base mutation detection method that is suitable for the detection of somatic cell mutations is sought. For the detection of somatic cell mutations, there is no need to elucidate the base sequence of the somatic cell mutation in detail, and it is sufficient to detect only the presence or absence of base mutations.

For example, methods for detecting the presence or absence of base mutations on the basis of differences between the melting curves for a wild-type sequence and a mutant sequence, by blocking the PCR amplification of the wild-type sequence by means of a synthetic nucleic acid PNA (Peptide Nucleic Acid) so that PCR amplification is performed specifically on the mutant sequence, then preparing a melting curve for the PCR product, is proposed (see Japanese Unexamined Patent Application, First Publication No. 2014-501533 (hereinafter, referred to as Patent Document 1), and Bjornar Gilje. et al., Journal of Molecular Diagnostics, 10, 325-331, 2008 (hereinafter, referred to as Non-Patent Document 1)).

However, in Patent Document 1 and Non-Patent Document 1, the presence or absence of base mutations is detected by using melting curves, so there are cases in which it is difficult to detect base mutations when only a few copies of nucleic acids having base mutations are present in a nucleic acid sample. Additionally, if there are single-nucleotide polymorphisms (SNP), base insertions, base deletions or the like aside from the base mutation to be detected, then a base mutation may be detected in error. Additionally, if the base mutation to be detected is an insertion or deletion of a single base, then there may be cases in which there is not a large difference in the melting curves between the wild-type and the mutant, making it difficult to distinguish therebetween.

### SUMMARY

Therefore, the present invention has the purpose of providing a base mutation detection method and kit that can detect a base mutation even when very few copies of nucleic acids having the base mutation are present in a nucleic acid sample. Additionally, the present invention has the purpose of providing a base mutation detection method and kit that can accurately detect a base mutation even when base mutations other than the base mutation that is to be detected are present in a nucleic acid sample. Furthermore, the present invention has the purpose of providing a base mutation detection method and kit that can detect a base mutation even when the base mutation to be detected is the insertion or deletion of a single base. The present invention also has the purpose of providing a method of sequence-specifically limiting PCR amplification in a nucleic acid sample.

The present invention is as described below.

A method for detecting a base mutation according to a first embodiment of the present invention is a detection method of a base mutation in a target base sequence of a nucleic acid sample, including: performing a PCR reaction with the nucleic acid sample as a template, using a primer set capable of amplifying, by PCR, an amplification target region including the target base sequence; a blocker nucleic acid fragment having a base sequence complementary to the target base sequence and including at least one residue which is a synthetic nucleic acid; and a probe that hybridizes to a region, in the amplification target region, closer to a 5′ end of the amplification target region than the target base sequence in the same chain as the target base sequence, and that has a fluorescent substance on one of a 5′ end and a 3′ end of the probe and a quenching substance on the other of the 5′ end and the 3′ end of the probe; and measuring an amplification amount of the template in the PCR reaction by detecting fluorescence from the probe.

In the first embodiment, the synthetic nucleic acid may be a BNA.

In the first embodiment, each of a melting temperature of the blocker nucleic acid fragment and a melting temperature of the probe may be higher than a melting temperature of the primer set.

In the first embodiment, the target base sequence may be a wild-type base sequence.

In the first embodiment, when performing the PCR reaction, a PCR reaction using a standard nucleic acid as a template, the standard nucleic acid having a wild-type base sequence, may be performed: and if a nucleic acid amplification amount when using the nucleic acid sample as the template is greater than a nucleic acid amplification amount when using the standard nucleic acid as the template, it may be determined that a base mutation is present in the target base sequence of the nucleic acid sample.

In the first embodiment, the target base sequence may be a base sequence of a KRAS, NRAS, BRAF, EGFR or PIK3CA gene.

A kit according to a second embodiment of the present invention is a kit for detecting a base mutation in a target base sequence of a nucleic acid sample, the kit including: a primer set capable of amplifying, by PCR, an amplification target region including the target base sequence; a blocker nucleic acid fragment having a base sequence complementary to the target base sequence and including at least one residue which is a synthetic nucleic acid; and a probe that hybridizes to a region, in the amplification target region, closer to a 5′ end of the amplification target region than the target base sequence in the same chain as the target base sequence, and that has a fluorescent substance on one of a 5′ end and a 3′ end of the probe and a quenching substance on the other of the 5′ end and the 3′ end of the probe.

In the second embodiment, the synthetic nucleic acid may be a BNA.

In the second embodiment, each of a melting temperature of the blocker nucleic acid fragment and a melting temperature of the probe may be higher than a melting temperature of the primer set.

In the second embodiment, the target base sequence is a base sequence of a KRAS, NRAS, BRAF, EGFR or PIK3CA gene.

A method for limiting PCR amplification of a nucleic acid sample having a target base sequence according to a third embodiment of the present invention includes preparing a primer set capable of amplifying, by PCR, an amplification target region including the target base sequence of the nucleic acid sample: and performing a PCR reaction with the nucleic acid sample as a template, using a blocker nucleic acid fragment having a base sequence complementary to the target base sequence and including at least one residue which is a synthetic nucleic acid.

In the third embodiment, the synthetic nucleic acid may be a BNA.

In the third embodiment, a melting temperature of the blocker nucleic acid fragment may be higher than a melting temperature of the primer set.

With the detection method and kit of the base mutation according to the above-mentioned embodiments of the present invention, it is possible to detect a base mutation even when very few copies of nucleic acids having the base mutation are present in a nucleic acid sample. Additionally, with the detection method and kit of the base mutation according to the above-mentioned embodiments of the present invention, a base mutation can be accurately detected even if base mutations other than the base mutation to be detected are present in the nucleic acid sample. Additionally, with the detection method and kit of the base mutation according to the above-mentioned embodiments of the present invention, a base mutation can be detected even if the base mutation to be detected is the insertion or deletion of a single base. Additionally, according to the above-mentioned embodiments of the present invention, it is possible to provide a method for sequence-specifically limiting PCR amplification in a nucleic acid sample.

### BRIEF DESCRIPTION OF THE DRAWINGS

FIG. 1 is a schematic diagram for explaining a base mutation detection method according to a first embodiment of the present invention.

FIG. 2 is a graph showing the results of detection of a base mutation in codons 12 and 13 in the KRAS gene.

FIG. 3 is a graph showing the results of detection of a base mutation in codon 61 in the KRAS gene.

FIG. 4 is a graph showing the results of detection of a base mutation in codon 146 in the KRAS gene.

FIG. 5 is a graph showing the results of detection of a base mutation in codons 12 and 13 in the KRAS gene.

FIG. 6 is a graph showing the results of detection of a base mutation in codon 61 in the KRAS gene.

### DETAILED DESCRIPTION OF THE PREFERRED EMBODIMENTS

[Base Mutation Detection Method]

The detection method of a base mutation in a target base sequence in a nucleic acid sample according to a first embodiment of the present invention includes using a primer set, a blocker nucleic acid fragment and a probe to perform a PCR reaction with the nucleic acid sample as the template. Furthermore, by detecting fluorescence from the probe, the amplification amount of the template due to the PCR reaction is measured. The primer set is capable of amplifying an amplification target region which includes the target base sequence by PCR. The blocker nucleic acid fragment has a base sequence that is complementary to the target base sequence, and includes at least one residue which is a synthetic nucleic acid. The probe has a fluorescent substance on one of the 5′ end and the 3′ end, and a quenching substance on the other of the 5′ end and the 3′ end, and hybridizes to a region, in the amplification target region, lying on the same chain as the target base sequence and lying closer to the 5′ end of the amplification target region than does the target base sequence.

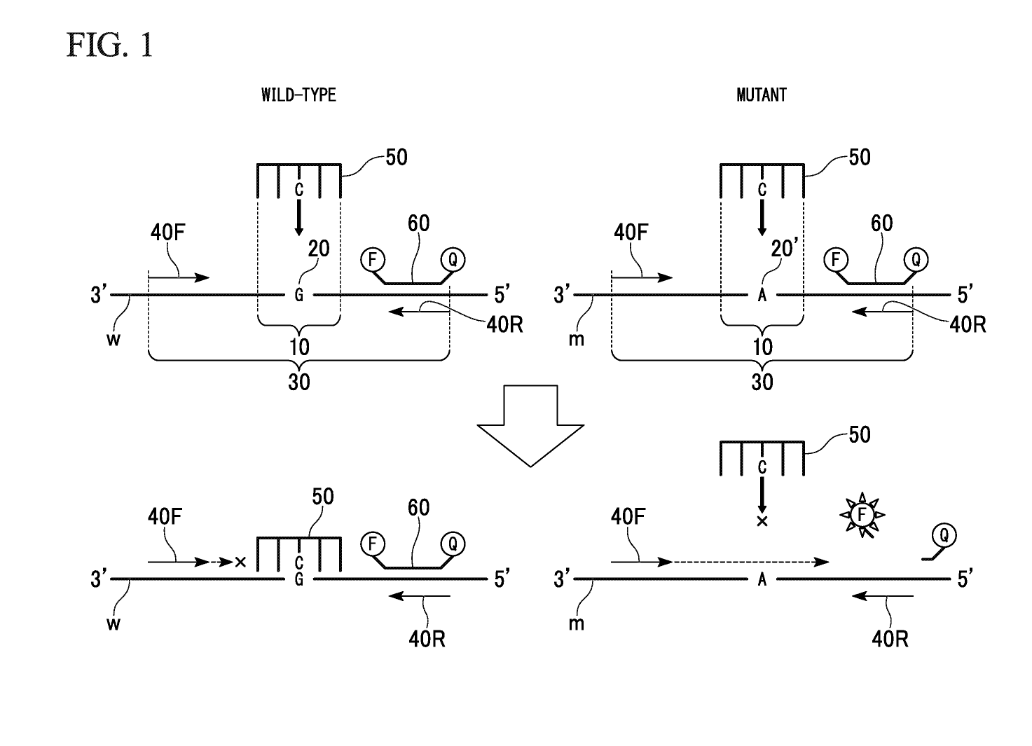

First, a summary of the base mutation detection method of the present embodiment will be described. FIG. 1 is a schematic diagram for explaining the base mutation detection method according to the present embodiment. Here, the case in which a base mutation 20′ (an adenine residue in FIG. 1) in a target base sequence 10 is to be detected, as shown in FIG. 1, will be explained. The residue corresponding to base mutation 20′ in the wild-type sequence is the residue 20 (a guanine residue in FIG. 1). In the present description, the region of the nucleic acid sample including the target base sequence (the part including the target base sequence) will be referred to as the target base sequence. Additionally, in the present description, a “base” may refer to a purine base or a pyrimidine base, such as adenine, guanine, thymine or cytosine.

Additionally, a “residue” refers to a unit corresponding to a nucleotide in the case of DNA. In other words, in the present description, a “base” refers to the base moiety, or a moiety corresponding thereto, of a residue comprising “a base, a sugar and a phosphoric acid”, in the case of DNA. Additionally, a “residue” refers to a unit comprising “a base, a sugar and a phosphoric acid”, or a unit corresponding thereto, in the case of DNA.

First, a PCR reaction is performed in the presence of a primer 40F and a primer 40R, a blocker nucleic acid fragment 50 and a probe 60. The primer 40F and the primer 40R form a primer set that can amplify an amplification target region 30 including the target base sequence 10 by PCR, using the nucleic acid sample w (wild-type) and the nucleic acid sample m (mutant) as templates. The blocker nucleic acid fragment 50 has a base sequence that is complementary to the target base sequence 10. The probe 60 has a fluorescent substance on one of the 5′ end and the 3′ end, and a quenching substance on the other of the 5′ end and the 3′ end, and hybridizes to a region, in the amplification target region 30, lying on the same chain as the target base sequence 10 and lying closer to the 5′ end than does the target base sequence 10.

Of the residues forming the blocker nucleic acid fragment 50, at least one residue is a synthetic nucleic acid.

Additionally, the probe 60 has a base sequence that is complementary to the template, and a fluorescent substance F is linked to one end of the probe 60, while a quenching substance Q is linked to the other end.

Additionally, each of the melting temperature of the blocker nucleic acid fragment 50 and the melting temperature of the probe 60 is preferably higher than the melting temperature of the primer 40F and the primer 40R. As a result, during the annealing step in the PCR reaction, the blocker nucleic acid fragment 50 and the probe 60 can be made to bind to the templates before the primer 40F and the primer 40R, and the base mutation detection precision can be improved.

In the PCR reaction, the reaction solution is first heated to approximately 90 to 99° C. to denature the nucleic acid. Next, the temperature of the reaction solution is lowered to approximately 50 to 60° C. and annealing is performed. In this case, if the melting temperatures of the blocker nucleic acid fragment 50 and the probe 60 are higher than the melting temperatures of the primer 40F and the primer 40R, then the blocker nucleic acid fragment 50 and the probe 60 bind to the templates before the primer 40F and the primer 40R. At this time, the blocker nucleic acid fragment 50 has a wild-type base sequence, and therefore binds to the wild-type template w. However, it does not bind to the mutant template m having the base mutation 20′. Thereafter, the primer 40F and the primer 40R bind to the templates.

Next, the temperature of the reaction solution is adjusted to approximately 60 to 75° C., and a primer elongation reaction is performed by using a Taq polymerase. In this case, a blocker nucleic acid fragment 50 is bound to the wild-type template w, so the primer elongation reaction is disrupted. On the other hand, the blocker nucleic acid fragment 50 is not bound to the mutant template m, so the primer elongation reaction proceeds.

Eventually, the primer elongation reaches the binding site of the probe 60. At this point, the probe is degraded by the 5′-to-3′ exonuclease activity of the Taq polymerase. As a result, the fluorescent substance F comes free from the quenching substance Q and begins to fluoresce.

When the PCR reaction is allowed to proceed by repeating the cycle of denaturation, annealing and elongation, the PCR amplification of the wild-type template w is limited, and PCR amplification of the mutant template m is predominantly performed. Additionally, the amount of the fluorescent substance F that is present increases in proportion to the PCR amplification rate of the template m.

Therefore, the PCR amplification rate of the template m can be measured by measuring the amount of fluorescence from the fluorescent substance R As a result, the presence of a base mutant 20′ can be detected in the target base sequence 10 of the nucleic acid sample. The above-mentioned limitation of the PCR amplification is efficiently performed due to the blocker nucleic acid fragment 50 containing at least one residue which is a synthetic nucleic acid. Specific synthetic nucleic acids are discussed below. The position of the synthetic nucleic acid may be that of any of the residues constituting the blocker nucleic acid fragment 50.

According to the method of the present embodiment, it is possible to detect base mutations even if very few copies of nucleic acids having the base mutation are present in a nucleic acid sample, by suppressing the PCR amplification of the wild-type template w by means of a blocker nucleic acid fragment 50.

Additionally, the base mutation in the target base sequence 10 can be accurately detected even if base mutations are present in regions of the amplification target region 30 other than the target base sequence 10. Additionally, a base mutation can be detected even if the base mutation is the insertion or deletion of a single base in the target base sequence 10. Additionally, even when there is a new base mutation, if it is within the range of the target base sequence 10, the base mutation can be detected without modifying the designs of the primer 40F, the primer 40R, the blocker nucleic acid fragment 50 and the probe 60.

Additionally, it is also possible to search for new base sequences by modifying the blocker nucleic acid fragment 50 (target base sequence 10).

Thus, according to the method of the present embodiment, it is possible to determine not only genotypes of somatic cell mutation genes of which very few copies are present, but also to adapt the method to mutations that may be newly discovered in the future. Therefore, the method of the present embodiment is also useful in personalized medicine, such as in the early discovery of cancer genes.

Next, the base mutation detection method according to the present embodiment will be explained in further detail.

(Base Mutation)

In the present description, base mutations refer to differences in the base sequences of genes that are present between individuals of the same biological species. According to the detection method of the present embodiment, it is possible to detect not only hereditary base mutations such as SNPs and microsatellite polymorphisms, but also acquired base mutations such as somatic cell mutations or the like caused by the substitution, deletion or insertion of one or more bases in a base sequence.

(Nucleic Acid Sample)

In the present embodiment, the nucleic acid sample may be a sample including genomic DNA from a test subject. Alternatively, the sample may contain an amplification product obtained by using a genomic DNA as a template and amplifying a region containing the target base mutation site, by PCR or the like, beforehand.

In the present embodiment, there may be just one or multiple target base sequences 10.

When there are multiple target base sequences 10, it is possible to carry out simultaneous PCR amplification (known as “multiplex PCR” or “mPCR”) of multiple amplification target regions 30, including each target base sequence 10, using multiple pairs of primer sets, with the nucleic acid sample as the template, and then to use the PCR amplification products thereof as nucleic acid samples to detect the base mutation in each target base sequence. As a result, even if the nucleic acid sample is small, for example, it is possible to increase the sample in order to detect the base mutations.

(Target Base Sequence)

The target base sequence 10 may be a wild-type base sequence. The blocker nucleic acid fragment 50 has a base sequence that is complementary to the target base sequence 10. Therefore, in this case, it is possible to limit PCR amplification of the wild-type template w, and to prioritize the PCR amplification of the mutant template m. As a result thereof, even if the amount of the mutant template m present in the nucleic acid sample is small, base mutations can be easily detected.

While the target base sequence is not particularly limited, it may, for example, be the base sequence of the KRAS, NRAS, BRAF, EGFR or PIK3CA gene. Mutations in these genes have been reported to be associated with cancer, particularly with colon cancer.

Additionally, for example, while anti-EGFR antibody is a therapeutic agent for cancer, it is known that the effects of anti-EGFR antibody cannot be obtained in the case of colon cancer involving mutations in codons 12 and 13 in the KRAS gene. Additionally, for example, in colon cancer, it has been reported that exon 2 in the KRAS gene may be of the wild-type, but about 10% of such patients have a mutation in the BRAF gene, in which case the prognosis is poor. Thus, detecting the presence or absence of somatic cell mutations in the above-described genes is very useful in choosing treatment methods of diseases.

(Melting Temperature)

The melting temperature (Tm) is the temperature at which 50% of the DNA molecules are denatured and become single strands. The melting temperature can be determined based on changes in the light absorbance of nucleic acids. While the bases constituting nucleic acids have strong absorption in the vicinity of wavelengths of 260 nm, in double-stranded nucleic acids, stacking interactions can cause the light absorbance of the nucleic acid overall to be lower than total absorbance of the individual bases. However, if a double-stranded nucleic acid solution is heated and the hydrogen bonds are severed, the individual bases will become free and begin absorbing light, thereby increasing the light absorbance compared to double-stranded nucleic acids. Therefore, upon measuring the light absorbance while changing the temperature of a nucleic acid, then plotting temperature on the horizontal axis and the absorbance on the vertical axis, a sigmoid curve is obtained. The melting temperature corresponds to the temperature at the inflection point of this curve, and can be defined as the temperature when the total increase in the light absorbance is 50%.

(Blocker Nucleic Acid Fragment)

The blocker nucleic acid fragment 50 includes at least one residue which is a synthetic nucleic acid.

Examples of synthetic nucleic acids that are currently known include nucleic acids modified by nucleosides such as peptide nucleic acids (PNA), bridged nucleic acids (BNA), glycol nucleic acids (GNA), threose nucleic acids (TNA), ENA (2′-O,4′-C-ethylene-bridged nucleic acids) and nucleoside enantiomers (e.g. β-L-deoxynucleoside for β-D-deoxynucleoside).

In the present embodiment, among the above, the synthetic nucleic acid is preferably BNA.

For example, PNA oligonucleotides have problems such as the difficulty of synthesizing structures of long chain length due to the specificity in the structures thereof, and the fact that they have extremely low solubility in water depending on the base sequence. On the other hand, BNA oligonucleotides do not have such problems, and are more suitable substances for use as the blocker nucleic acid fragment in the present embodiment.

Additionally, BNAs have a high Tm value compared to other synthetic nucleic acids, and are therefore more suitable for use as blockers. If the Tm is high, for example, even during a reaction in a high-temperature region in a nucleic acid amplification reaction (PCR), such as during the step of denaturing a nucleic acid by heating the reaction solution to about 90 to 99° C., the blocker nucleic acid fragment will not easily separate from the linking portion with the template w, so the PCR amplification of the template w can be more effectively limited.

The present inventors discovered that a blocker nucleic acid fragment 50 having at least one residue that is a BNA exhibits greatly improved PCR amplification limitation efficiency. When a nucleic acid fragment in which all of the residues are BNAs is used as the blocker nucleic acid fragment 50, there is a risk of blocking regions that do not completely match, such as mutant sequences and other regions with similar sequences.

BNA is a collective term for bridged nucleic acids. Known examples of BNAs include 2′,4′-BNA, 3′,4′-BNA, 2′-deoxy-3′-N-3′,4′-BNA, 3′-N-2′,4′-BNA, which are nucleotides having two cyclic structures wherein the oxygen atom at the 2′ position and the carbon atom at the 4′ position in ribose are bound by methylene; 2′-4′-BNA^{NC}, in which the O at the 2 position and the C at the 4 position in a furanose ring are bridged by —NRCH₂— (where R is a methyl group); and 2′-4′-BNA^{COC}, in which the O at the 2 position and the C at the 4 position in a furanose ring are bridged by —CH₂OCH₂—.

While the BNA contained in the blocker nucleic acid fragment 50 may be any of the above-mentioned BNAs, 2′,4′-BNA is preferred. Additionally, the length of the blocker nucleic acid fragment 50 is not particularly limited as long as the melting temperature is higher than that of the primer 40F and the primer 40R.

(Probe)

The probe that is used in the present embodiment is an oligonucleotide having a fluorescent substance on one of the 5′ end and the 3′ end, and having a quenching substance on the other of the 5′ end and the 3′ end. As the probe, a TaqMan (registered trademark) probe may be used. Examples of the fluorescent substance that may be used include FAM, Yakima Yellow (registered trademark), fluorescein, FITC and VIC (registered trademark). Additionally, examples of the quenching substance that may be used include TAMRA and the like.

In the present embodiment, during the annealing step of the PCR reaction, the primer 40F, the blocker nucleic acid fragment 50 and the probe 60 must be bound, in the stated sequence, from the 3′ side to the 5′ side of the same chain on the template w. As a result, on the template w. PCR amplification of the template w is limited and the degradation of the probe 60 is limited. Additionally, in the template m, the blocker nucleic acid fragment 50 does not bind, so the elongation of the primer 40F causes degradation of the probe 60, thereby freeing standard quantities of the fluorescent substance F.

### (Template Amplification Amount Measurement Step)

In the present embodiment, the PCR amplification amount of a template can be measured by measuring the amount of fluorescence from the fluorescent substance F that is freed by degradation of the probe 60. The fluorescence measurement is preferably performed in real-time, simultaneously with the PCR reaction. When measuring fluorescence in real-time in this way, the measurements can be made using various devices that are used for real-time quantitative PCR.

When detecting base mutations, it is preferable to provide a nucleic acid sample as a control. This allows base mutations to be more accurately detected. As a control, for example, a nucleic acid sample (standard nucleic acid) having a wild-type base sequence may be used. In this case, it is possible to determine that a base mutation is present in the target base sequence in the nucleic acid sample if the PCR amplification amount of the template when using a nucleic acid sample as a template is greater than the PCR amplification amount of a template when using the standard nucleic acid as the template.

In other words, in the method for detecting base mutations in a target base sequence of a nucleic acid sample according to a first embodiment of the present invention, a PCR reaction is performed with a wild-type base sequence as the target base sequence, using a primer set, a blocker nucleic acid fragment and a probe, and using the aforementioned nucleic acid sample and a standard nucleic acid having a wild-type base sequence as templates. The primer set is capable of amplifying an amplification target region including the target base sequence by PCR. The blocker nucleic acid fragment has a base sequence that is complementary to the target base sequence, and includes at least one residue which is a synthetic nucleic acid. The probe has a fluorescent substance on one of the 5′ end and the 3′ end, and a quenching substance on the other of the 5′ end and the 3′ end, and hybridizes to a region, in the amplification target region, lying on the same chain as the target base sequence and lying closer to the 5′ end of the amplification target region than the target base sequence. Furthermore, the template amplification amount in the PCR reaction is measured by detecting the fluorescence from the probe. Furthermore, if the nucleic acid amplification amount when using the nucleic acid sample as the template is greater than the nucleic acid amplification amount obtained when using the standard nucleic acid as the template, it is determined that a base mutation is present in the target base sequence of the nucleic acid sample.

[Kit for Detecting Base Mutations].

The kit for detecting base mutations in a target base sequence of a nucleic acid sample according to the second embodiment of the present invention includes a primer set, a blocker nucleic acid fragment and a probe. The primer set is capable of amplifying an amplification target region including the target base sequence by PCR. The blocker nucleic acid fragment has a base sequence that is complementary to the target base sequence, and includes at least one residue which is a synthetic nucleic acid. The probe has a fluorescent substance on one of the 5′ end and the 3′ end of the probe, and a quenching substance on the other of the 5′ end and the 3′ end of the probe, and hybridizes to a region, in the amplification target region, lying on the same chain as the target base sequence and lying closer to the 5′ end of the amplification target region than the target base sequence.

The kit according to the present embodiment can be favorably used in the above-described base mutation detection method. The specific forms of the blocker nucleic acid fragment and the probe are the same as those mentioned above.

According to the kit of the present embodiment, it is possible to detect a base mutation in a nucleic acid sample, even when only a few copies of the template (e.g., a nucleic acid having the base mutation) to be detected are present, by limiting PCR amplification of a template (e.g., a nucleic acid having a wild-type base sequence) that is not a detection target, by means of a blocker nucleic acid fragment.

Additionally, a base mutation can be accurately detected in the target base sequence even if base mutations are present in regions of the amplification target region other than the target base sequence. Additionally, a base mutation in the target base sequence can be detected even if the base mutation is the insertion or deletion of a single base in the target base sequence. Additionally, even when there is a new base mutation, the base mutation can be detected as long as it is within the range of the target base sequence.

In the kit according to the present embodiment, the synthetic nucleic acid is preferably a BNA in view of the increased PCR amplification limitation efficiency by the blocker nucleic acid fragment. The preferred BNA is the same as that mentioned above. Additionally, each of the melting temperature of the blocker nucleic acid fragment and the melting temperature of the probe is preferably higher than the melting temperature of the primer set. As a result, it is possible to make the blocker nucleic acid fragment and the probe bind to the template before the primers during the annealing step in the PCR reaction, thereby improving the base mutation detection accuracy.

The kit according to the present embodiment may have, as a target base sequence, the base sequence of the KRAS, NRAS, BRAF, EGFR or PIK3CA gene. Such kits are useful for diagnosing and choosing treatment methods for cancers such as colon cancer.

[Method for Limiting PCR Amplification in Nucleic Acid Sample]

The method for limiting PCR amplification in a nucleic acid sample having a target base sequence according to the third embodiment of the present invention uses a primer set that is capable of amplifying an amplification target region including the target base sequence in the nucleic acid sample by PCR. The primer set is capable of PCR amplification of an amplification target region including the target base sequence in the nucleic acid sample. Furthermore, the PCR reaction using the nucleic acid sample as the template is performed in the presence of a blocker nucleic acid fragment having a base sequence that is complementary to the target base sequence, and including at least one residue which is a synthetic nucleic acid.

According to the method of the present embodiment, it is possible to specifically limit the PCR amplification of a template having a specific base sequence. The specific forms of the blocker nucleic acid fragment are the same as those mentioned above.

In the method of the present embodiment, the synthetic nucleic acid is preferably a BNA in view of the increased PCR amplification limitation efficiency by the blocker nucleic acid fragment. The preferred BNAs are the same as those mentioned above. Additionally, the melting temperature of the blocker nucleic acid fragment is preferably higher than the melting temperature of the primer set. As a result, it is possible to make the blocker nucleic acid fragment bind to the template before the primers during the annealing step in the PCR reaction, thereby further improving limitation efficiency of the PCR amplification by the blocker nucleic acid fragment.

### EXAMPLES

Herebelow, the present invention will be explained by referring to experimental examples, but the present invention is not to be construed as being limited to the following experimental examples.

### Experimental Example 1

Base mutations were detected in codons 12 and 13, codon 61 and codon 146 of KRAS, which is a cancer gene.

The reagents shown in Table 1 were used to prepare reaction solutions for PCR reactions so as to have the compositions in Table 2.

Additionally, a reaction solution to which a blocker nucleic acid fragment was added and a reaction solution to which a blocker nucleic acid fragment was not added were prepared.

When not adding a blocker nucleic acid fragment, the same volume of water was added. BNA (2′,4′-BNA) was used as the synthetic nucleic acid in the blocker nucleic acid fragment.

Additionally, if all of the residues of the blocker nucleic acid fragment were to be composed of BNA, then there is a possibility that the high binding force thereof would limit the PCR amplification of not only the wild-type template, but also that of the mutant template. Therefore, normal nucleotide residues were randomly included in the blocker nucleic acid fragment. Of the base sequences for the blocker nucleic acid fragments shown in Table 1, the residues that are in boldface and underlined indicate BNA, and those in normal type indicate normal nucleotide residues.

Additionally, in the probe, FAM was used as the fluorescent substance and TAMRA was used as the quenching substance.

Additionally, human genomic DNA were used in the nucleic acid samples. The specific content of the nucleic acid samples will be described below.

TABLE 1

| Detected | | |
|---|---|---|
| mutation | | |
| site | Reagent | Base Sequence (5′ to 3′) |
| KRAS | Forward | CTGAATATAAACTTGTGGTAGTTGG |
| codons | primer | (SEQ ID NO: 1) |
| 12 and | Reverse | GTCCTGCACCAGTAATATGC |
| 13 | primer | (SEQ ID NO: 1) |
| | Blocker | CCTACGCCACC |
| | nucleic | (SEQ ID NO: 3) |
| | acid | |
| | fragment | |
| | Probe | (FAM)TGCCTTGACGATACAGCTAA |
| | | TTCAGAA(TAMRA) |
| | | (SEQ ID NO: 4) |
| KRAS | Forward | ACTGTGTTTCTCCCTTCTCA |
| codon | primer | (SEQ ID NO: 5) |
| 61 | Reverse | CAGTCCTCATGTACTGGTCC |
| | primer | (SEQ ID NO: 6) |
| | Blocker | TCCTCTTGACCTGC |
| | nucleic | (SEQ ID NO: 7) |
| | acid | |
| | fragment | |
| | Probe | (FAM)TCCAAGAGACAGGTTTCTCCA |
| | | TCAA(TAMRA) |
| | | (SEQ ID NO: 8) |
| KRAs | Forward | GTGATTTGCCTTCTAGAACAGT |
| codon | primer | (SEQ ID NO: 9) |
| 146 | Reverse | CAGAAAACAGATCTGTATTTATTTCAG |
| | | (SEQ ID NO: 10) |
| | Blocker | TCTTTGCTGATG |
| | nucleic | (SEQ ID NO: 11) |
| | acid | |
| | fragment | |
| | Probe | (FAM)AGGCTCAGGACTTAGCAAGAAG |
| | | TTATGG(TAMRA) |
| | | (SEQ ID NO: 12) |

TABLE 2

| | Reagent |
|---|---|
| Reagent | Amonnt |
| TaqMan (registered trademark) Gene | 25 μL |
| Expression Master Mix (Life Technologies | |
| Japan) | |
| 10 μM Forward Primer (SIGMA) | 2 μL |
| 10 μM Reverse Primer (SIGMA) | 2 μL |
| 5 μM Probe (Life Technologies Japan) | 2.5 μL |
| 5 μM Blocker Nucleic Acid Fragment | 1 μL |
| (Gene Design) | |
| 10 ng/μL Genomic DNA | 3 μL |
| Water | 14.5 μL |
| Total Reaction Solution Amount | 50 μL |

Next, the prepared reaction solutions for the PCR reaction were set in a real-time PCR analysis system (product name “CFX96”, Bio-Rad), held at 50° C. for 2 minutes, then held at 95° C. for 10 minutes to activate the enzymes in the TaqMan (registered trademark) Gene Expression Master Mix. Then, denaturation was performed by holding the solutions at 95° C. for 15 seconds, then annealing and elongation were performed by holding the solutions at 60° C. for 30 seconds. These cycles of 15 seconds at 95° C. and 30 seconds at 60° C. were repeated 40 times.

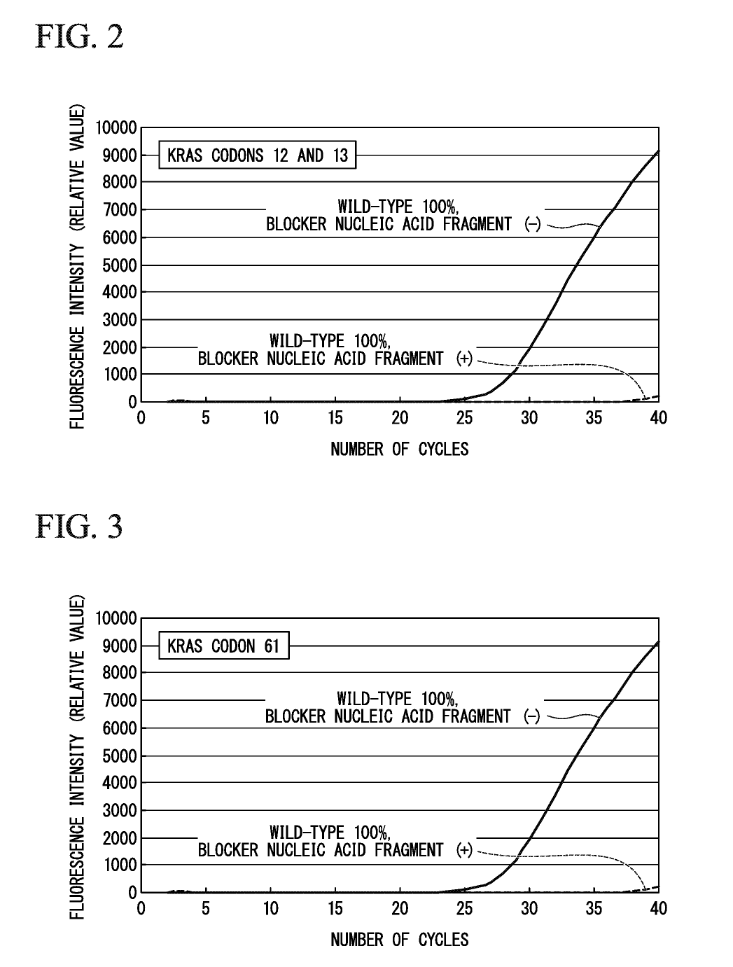

FIG. 2 is a graph showing the results for detection of a base mutation in codons 12 and 13 in the KRAS gene. The horizontal axis indicates the number of PCR cycles and the vertical axis indicates the intensity of the fluorescence from the probe. As the nucleic acid sample, human genomic DNA having a 100% wild-type base sequence for codons 12 and 13 in the KRAS gene was used.

The results of the real-time PCR reaction curves in the presence (indicated by “(+)” in FIG. 2) and absence (indicated by “(−)” in FIG. 2) of a blocker nucleic acid fragment showed that the PCR amplification of the template was limited in the presence of a blocker nucleic acid fragment. This result indicates that a base mutation is not present in codons 12 and 13 in the KRAS gene of the nucleic acid sample.

FIG. 3 is a graph showing the results of detection of a base mutation in codon 61 of the KRAS gene in the same manner as described above. As the nucleic acid sample, human genomic DNA having a 100% wild-type base sequence for codon 61 in the KRAS gene was used.

The results of the real-time PCR reaction curves in the presence (indicated by “(+)” in FIG. 3) and absence (indicated by “(−)” in FIG. 3) of a blocker nucleic acid fragment showed that the PCR amplification of the template was limited in the presence of a blocker nucleic acid fragment. This result indicates that a base mutation is not present in codon 61 in the KRAS gene of the nucleic acid sample.

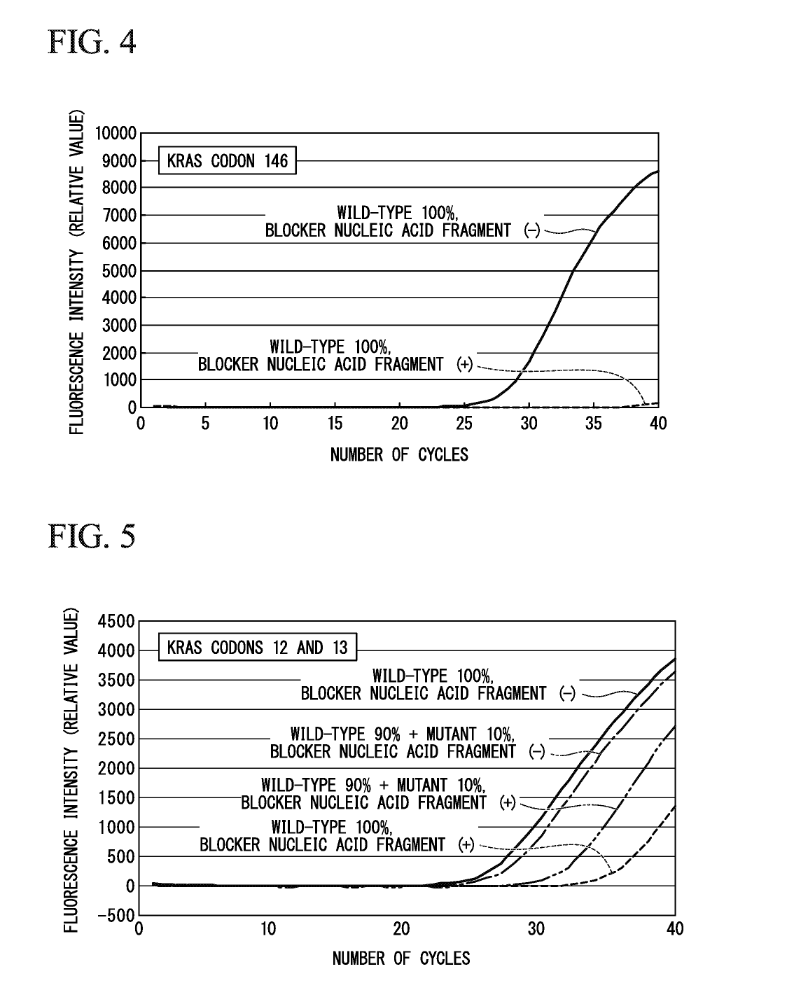

FIG. 4 is a graph showing the results of detection of a base mutation in codon 146 of the KRAS gene in the same manner as described above. As the nucleic acid sample, human genomic DNA having a 100% wild-type base sequence for codon 146 in the KRAS gene was used.

The results of the real-time PCR reaction curves in the presence (indicated by “(+)” in FIG. 4) and absence (indicated by “(−)” in FIG. 4) of a blocker nucleic acid fragment showed that the PCR amplification of the template was limited in the presence of a blocker nucleic acid fragment. This result indicates that a base mutation is not present in codon 146 in the KRAS gene of the nucleic acid sample.

### Experimental Example 2

A reaction solution for a PCR reaction was prepared in the same manner as Experimental Example 1, except that the nucleic acid sample was changed. The specific contents of the nucleic acid sample will be described below. Additionally, a reaction solution to which a blocker nucleic acid fragment was added and a reaction solution to which a blocker nucleic acid fragment was not added were prepared. When not adding a blocker nucleic acid fragment, the same volume of water was added.

FIG. 5 is a graph showing the results of detection of base mutations in codons 12 and 13 in the KRAS gene. As the nucleic acid sample, human genomic DNA having a 100% wild-type base sequence for codons 12 and 13 in the KRAS gene (indicated as “wild-type” in FIG. 5), or a mixture of 90% human genomic DNA having the above-mentioned wild-type sequence and 10% human genomic DNA having a G13D mutation (GGC to GAC) in codons 12 and 13 of the KRAS gene (indicated as “mutant” in FIG. 5) was used.

The results of the real-time PCR reaction curves in the presence (indicated by “(+)” in FIG. 5) and absence (indicated by “(−)” in FIG. 5) of a blocker nucleic acid fragment showed that, in the presence of a blocker nucleic acid fragment, the PCR amplification of the wild-type template was limited, and the PCR amplification was selectively performed on the mutant template.

Additionally, in the presence of the blocker nucleic acid fragment, the amplification amount in the nucleic acid sample containing the mutant template was greater than the amplification amount of the wild-type template. This result shows that a base mutation is present in codons 12 and 13 in the KRAS gene in the nucleic acid sample. Additionally, this result shows that a base mutation can be definitely detected if a mutant template is present as 10% of a nucleic acid sample.

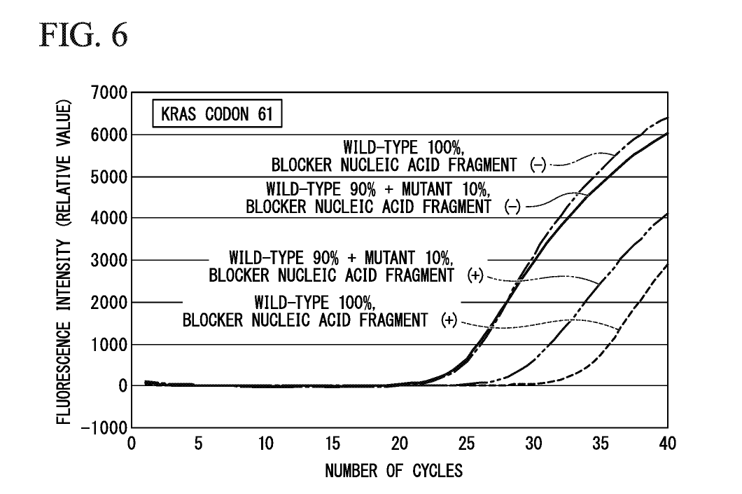

FIG. 6 is a graph showing the results of detection of a base mutation in codon 61 of the KRAS gene.

As the nucleic acid sample, human genomic DNA having a 100% wild-type base sequence for codon 61 in the KRAS gene (indicated as “wild-type” in FIG. 6), or a mixture of 90% human genomic DNA having the above-mentioned wild-type sequence and 10% human genomic DNA having a Q61H mutation (CAA to CAC) in codon 61 of the KRAS gene (indicated as “mutant” in FIG. 6) was used.

The results of the real-time PCR reaction curves in the presence (indicated by “(+)” in FIG. 6) and absence (indicated by “(−)” in FIG. 6) of a blocker nucleic acid fragment showed that, in the presence of a blocker nucleic acid fragment, the PCR amplification of the wild-type template was limited, and the PCR amplification was selectively performed on the mutant template.

Additionally, in the presence of the blocker nucleic acid fragment, the amplification amount of the nucleic acid sample containing the mutant template was greater than the amplification amount of the wild-type template. This result shows that a base mutation is present in codon 61 in the KRAS gene in the nucleic acid sample. Additionally, this result shows that a base mutation can be definitely detected if a mutant template is present as 10% of a nucleic acid sample.

With the base mutation detection method and kit according to the present invention, it is possible to detect a base mutation even when very few copies of nucleic acids having the base mutation are present in a nucleic acid sample. Additionally, a base mutation can be accurately detected even if base mutations other than the base mutation to be detected are present. Additionally, a base mutation can be detected even if the base mutation to be detected is the insertion or deletion of a single base. Additionally, according to the present invention, it is possible to provide a method for sequence-specifically limiting PCR amplification in a nucleic acid sample.

What is claimed is:

1. A detection method of a base mutation in a target base sequence of a nucleic acid sample, comprising:
performing a PCR reaction with the nucleic acid sample as a template, using a primer set capable of amplifying, by PCR, an amplification target region including the target base sequence; a blocker nucleic acid fragment having a base sequence complementary to the target base sequence and including at least one residue which is a synthetic nucleic acid; and a probe that hybridizes to a region, in the amplification target region, closer to a 5′ end of the amplification target region than the target base sequence in the same chain as the target base sequence, and that has a fluorescent substance on one of a 5′ end and a 3′ end of the probe and a quenching substance on the other of the 5′ end and the 3′ end of the probe; and
measuring an amplification amount of the template in the PCR reaction by detecting fluorescence from the probe.

2. The detection method according to claim 1, wherein
the synthetic nucleic acid is a BNA.

3. The detection method according to claim 1, wherein
each of a melting temperature of the blocker nucleic acid fragment and a melting temperature of the probe is higher than a melting temperature of the primer set.

4. The detection method according to claim 1, wherein
the target base sequence is a wild-type base sequence.

5. The detection method according to claim 4, wherein
when performing the PCR reaction, a PCR reaction using a standard nucleic acid as a template, the standard nucleic acid having a wild-type base sequence, is performed; and
if a nucleic acid amplification amount when using the nucleic acid sample as the template is greater than a nucleic acid amplification amount when using the standard nucleic acid as the template, it is determined that a base mutation is present in the target base sequence of the nucleic acid sample.

6. The detection method according to claim 1, wherein
the target base sequence is a base sequence of a KRAS, NRAS, BRAF, EGFR or PIK3CA gene.

7. A kit for detecting a base mutation in a target base sequence of a nucleic acid sample, the kit comprising:
a primer set capable of amplifying, by PCR, an amplification target region including the target base sequence;
a blocker nucleic acid fragment having a base sequence complementary to the target base sequence and including at least one residue which is a synthetic nucleic acid; and
a probe that hybridizes to a region, in the amplification target region, closer to a 5′ end of the amplification target region than the target base sequence in the same chain as the target base sequence, and that has a fluorescent substance on one of a 5′ end and a 3′ end of the probe and a quenching substance on the other of the 5′ end and the 3′ end of the probe.

8. The kit according to claim 7, wherein
the synthetic nucleic acid is a BNA.

9. The kit according to claim 7, wherein
each of a melting temperature of the blocker nucleic acid fragment and a melting temperature of the probe is higher than a melting temperature of the primer set.

10. The kit according to claim 7, wherein
the target base sequence is a base sequence of a KRAS, NRAS, BRAF, EGFR or PIK3CA gene.

11. A method for limiting PCR amplification of a nucleic acid sample having a target base sequence, comprising:
preparing a primer set capable of amplifying, by PCR, an amplification target region including the target base sequence of the nucleic acid sample; and
performing a PCR reaction with the nucleic acid sample as a template, using a blocker nucleic acid fragment having a base sequence complementary to the target base sequence and including at least one residue which is a synthetic nucleic acid.

12. The method according to claim 11, wherein
the synthetic nucleic acid is a BNA.

13. The method according to claim 12, wherein
a melting temperature of the blocker nucleic acid fragment is higher than a melting temperature of the primer set.
